(19) United States
(12) Patent Application Publication
(10) Pub. No.: US 2022/0047682 A1
(43) Pub. Date: Feb. 17, 2022

(54) COMPOSITIONS AND METHODS FOR BIODEGRADING ALCOHOL

(71) Applicants: Tami BAR, Metar (IL); Thomas KOEVARY, Jerusalem (IL)

(72) Inventors: Tami BAR, Metar (IL); Thomas KOEVARY, Jerusalem (IL)

(21) Appl. No.: 17/288,619

(22) PCT Filed: Oct. 24, 2019
(86) PCT No.: PCT/IL2019/051151

§ 371 Date: Apr. 26, 2021

Related U.S. Application Data
(60) Provisional application No. 62/750,862, filed on Oct. 26, 2018.
(60) Provisional application No. 62/758,722, filed on Nov. 12, 2018.

(51) Int. Cl. A61K 38/44; A61P 25/32

(52) U.S. Cl. CPC A61K 38/443; C12Y 101/01001; A61P 25/32

(57) ABSTRACT

The present invention provides a pharmaceutical composition containing 10 mg to about 100 g KRED and/or a long-acting alcohol dehydrogenase as an active ingredient and a pharmaceutically acceptable carrier. Moreover, provided herein methods for lowering blood alcohol level, methods for preventing a symptom or a risk arising from alcohol consumption and methods for treating a subject afflicted with alcoholism by the administration of the pharmaceutical composition of the invention.

### FIELD OF INVENTION

This invention is directed to; inter alia, compositions and methods for discarding alcohol in a physiological environment.

### BACKGROUND OF THE INVENTION

Alcohol Dehydrogenase (ADH) refers to a family of enzymes which catalyze the reversible oxidation of primary or secondary alcohols to aldehydes or ketones. ADH has many roles in the body; a major function is to catalyze the oxidation of ethanol (EtOH) to acetaldehyde as the first step of EtOH metabolism by the liver, using NAD+ or NADP+ as electron acceptors in the process.

There are more than 9 different forms of ADH produced by the human body and each has a specific role in different areas of the digestive tract though the enzyme has been found to be especially highly concentrated in the liver and kidney. The ability to break down alcohol using a specific treatment or formulation will be an imperative tool for clinical and pharmacological uses.

Alcohol abuse is a significant cause of accidents and death. Major alcohol related problems exist in almost every phase of human activity including recreation and the workplace. Chronic alcohol abuse leads to many serious disorders, most commonly liver cirrhosis. Twenty percent of emergency room visits in the United States, approximately 90 million visits, are alcohol related. Lethal blood ethanol concentrations are generally in the range of 0.25% and 1.50%. Ethanol overdose without complications leads to approximately 1000 deaths per year in the United States.

Ethanol is rapidly transported into the blood from the intestine, and is also transported into the blood from the stomach. Metabolism of ingested alcohol as measured by disappearance of ethanol from the blood, follows zero order kinetics above blood alcohol concentration (BAC) values of 2 mM. The linear rate of blood alcohol elimination is 2 to 5 mM/hour, accordingly, four to ten hours are required to remove most of the alcohol from the body.

As reviewed in Biochemistry and Pharmacology of Ethanol, vol. 1, (Majchrowicz, ed., Plenum Press, N.Y., 1979), ethanol is eliminated by respiration, excretion, and metabolism, ninety percent of which occurs in the liver. In general, liver alcohol dehydrogenase (LADH) metabolizes the majority of the ethanol. The alcohol metabolism rate is limited by the relatively low value of the LADH Michaelis constant (Km) and the NADH (nicotinamide adenine dinucleotide, reduced form) oxidation (regeneration) rate. The microsomal alcohol-oxidizing system (MAOS) located in microsomes of the smooth endoplasmic reticulum of hepatocytes is a second alcohol metabolism mechanism. This mechanism is dependent on regeneration of NADPH (nicotinamide adenine dinucleotide phosphate, reduced form). A third alcohol metabolizing mechanism depends on the enzyme catalase and hydrogen peroxide. This mechanism is thought to metabolize relatively little ethanol in vivo because of the need for hydrogen peroxide at the enzyme site. A gastric alcohol dehydrogenase present in stomach mucosa is a fourth alcohol metabolizing mechanism. The importance of this fourth mechanism of alcohol metabolism relative to the LADH mechanism is not clear at this time.

Only two methods are known to accelerate ethanol elimination from the body. Ingestion of fructose can increase the ethanol elimination rate by 0% to 25% greater than control values in some subjects. It is thought that fructose only increases NADH regeneration, thereby helping to maintain the LADH mechanism at its maximum intrinsic rate of ethanol metabolism. Dialysis of alcohol-containing blood using traditional kidney dialysis only slightly accelerates alcohol elimination.

In 1980, J. C. Commander published a M.S. thesis from Auburn University, Alabama, proposing that an enzyme system be used for the detoxification of alcohol. The study reported that ethanol diffused from the blood into the intestinal lumen when the blood ethanol concentration was greater than the intestinal ethanol concentration. Commander proposed using an enzyme system isolated from liver, which remains active in the intestine. Generally, the intestinal lumen pH is 6.0 to 8.0 and 80% of ingested ethanol is absorbed into the blood from the intestinal lumen. The multi-enzyme system which he tested in vitro included buffer salts containing bovine serum albumin (BSA), potassium ions, thiol groups (e.g. β-2-mercaptoethanol), NAD, NADH, acetaldehyde, alcohol dehydrogenase, and aldehyde dehydrogenase (ALDH) in various concentration.

In 1988, D. R. Whitmire published as his Ph.D. dissertation “Multi Enzyme System with Substrate Pumped NAD Recycling Applied to ethanol Detoxification of the Dog”. This dissertation reported the development of a method using yeast alcohol dehydrogenase (YADH) and yeast aldehyde dehydrogenase (YALDH) in an appropriate buffer to oxidize alcohol to acetate using lactate dehydrogenase (LDH) catalyzed pyruvate pumped NAD recycling. A second system using a cell-free extract of gluconobacter suboxydans in an appropriate buffer was also developed and shown to oxidize ethanol. A third system using YADH, YALDH in combination with glycerol dehydrogenase (GDH) as the recycle enzyme was also described. Problems with gastric pH deactivation, proteolytic degradation, and bile salt inactivation of the enzymes were overcome using protease inhibitors, pepstatin, and a sucrose-phosphate-dithiothreitol buffer. However, in vivo use of these enzyme systems posed significant problems: The system had a high pyruvate requirement with is not normally present in the intestine in large quantities; sucrose buffer (50% w/v) was required to stabilize the YALDH against bile salt actions; pyruvate and lactate are univalent ions of salts which yield two moles of solute for each mole of salt; high sucrose concentration and high salt concentration caused the enzyme system to be hyperosmolar; and lactate produced by the recycling reaction can potentially lead to lactic acidosis. Accordingly, while this approach demonstrated the theoretical feasibility of using an orally administered formulation of enzymes to rapidly oxidize ethanol which exsorbed into the intestine, because of the significant problems enumerated above, it does not provide a practical, commercially acceptable means for accelerating ethanol elimination from the body.

### SUMMARY OF THE INVENTION

In one embodiment, the present invention provides a protein comprising an amino acid sequence encoding ADH/KRED bound to at least one long-acting molecule or complexing molecule.

In one embodiment, the present invention provides a pharmaceutical composition, comprising 10 mg to 100 g of KRED and a pharmaceutically acceptable carrier.

In one embodiment, the present invention provides a pharmaceutical composition, comprising the protein comprising an amino acid sequence encoding ADH/KRED bound to at least one long-acting molecule and a pharmaceutically acceptable carrier.

In another embodiment, the present invention further provides a method for lowering blood alcohol in a subject in need thereof, comprising administering to said subject a composition comprising an effective amount of: a protein comprising an amino acid sequence encoding ADH/KRED bound to at least one long-acting molecule, thereby lowering blood alcohol in a subject in need thereof.

In another embodiment, the present invention further provides a method for lowering blood alcohol in a subject in need thereof, comprising administering to said subject a composition comprising an effective amount of KRED, thereby lowering blood alcohol in a subject in need thereof.

In another embodiment, the present invention further provides a method for preventing a symptom or a risk arising from alcohol consumption in a subject in need thereof, comprising administering to the subject a composition comprising an effective amount of KRED, thereby preventing a symptom or a risk arising from alcohol consumption in a subject in need thereof.

In another embodiment, the present invention further provides that the methods of the invention include administering before alcohol consumption, after alcohol consumption, or both before and after alcohol consumption

In another embodiment, the present invention further provides a method for treating a subject afflicted with alcoholism, comprising administering to the subject a composition comprising an effective amount of KRED, thereby treating a subject afflicted with alcoholism.

In another embodiment, the present invention further provides a method for treating a subject afflicted with alcoholism or alcohol poisoning, comprising administering to the subject a composition comprising an effective amount of: a protein comprising an amino acid sequence encoding KRED and/or ADH/KRED bound to at least one long-acting molecule, thereby treating a subject afflicted with alcoholism or alcohol poisoning.

### BRIEF DESCRIPTION OF THE DRAWINGS

FIG. 1. Is a bar graph showing the effect of EtOH dose (0.05 g/ml-0.2 g/ml) on Open Field activity (Activity Box).

FIG. 2. Is a bar graph showing the effect of EtOH (0.5 g/kg) and ADH (10, 100 and 500 mg/kg) (cycles 1 &2) on distance traveled in Activity Box tests during a 30 min of trial.

FIG. 3. Is a bar graph showing the ability to rescue and prevent the neurological effects of EtOH before and after consumption.

FIG. 4. Is a bar graph showing the beam walking ability of mice after treatment with EtOH.

FIG. 5. Is a bar graph showing the rescue effect of ADH on beam walking ability of mice treated with EtOH.

FIG. 6. Is a bar graph showing that ADH I was effective by administration before and after EtOH administration.

FIG. 7. Is a bar graph showing the ex vivo evaluation of the effect of ADH on EtOH concentration in human plasma (3 volunteers, 30 minutes after ADH addition).

FIGS. 8-10. Are bar graphs showing the Ex vivo evaluation of the effect of ADH on EtOH concentration in human plasma (3 volunteers 30 min after ADH addition).

FIGS. 11A-11D. Are graphs showing evaluation of the effect of ADHs on acute EtOH intoxication at various doses in mice.

### DETAILED DESCRIPTION OF THE INVENTION

In one embodiment, the present invention provides an alcohol dehydrogenase (ADH) having enhanced longevity compared to the wild-type ADH counterpart. In another embodiment, the present invention provides an alcohol dehydrogenase (ADH) having enhanced in vivo potency compared to the wild-type ADH counterpart. In another embodiment, the present invention provides long-acting alcohol dehydrogenase (ADH) compared to the wild-type ADH counterpart. In another embodiment, the ADH to be modified according to the invention has the Enzyme Commission number EC 1.1.1.1. In one embodiment, the present invention provides a composition, comprising 10 mg to 100 g of KRED. In one embodiment, the present invention provides a composition, comprising 10 mg to 10 g of KRED. In another embodiment, KRED is a Codexis KRED.

In another embodiment, an ADH comprises KRED of the invention oxidizes an alcohol substrate to the corresponding ketone/aldehyde product in the presence of a co-factor such as nicotinamide adenine dinucleotide (NADH) or reduced nicotinamide adenine dinucleotide phosphate (NADPH), and nicotinamide adenine dinucleotide (NAD) or nicotinamide adenine dinucleotide phosphate (NADP).

In another embodiment, long-acting alcohol dehydrogenase (ADH) of the invention compared to the wild-type ADH has extended circulatory half-lives. long-acting alcohol dehydrogenase (ADH) of the invention compared to the wild-type ADH has enhanced potency.

In another embodiment, long-acting alcohol dehydrogenase (ADH) of the invention compared to the wild-type ADH counterpart has a higher area under the curve (AUC) value within the blood and/or serum. In another embodiment, long-acting alcohol dehydrogenase (ADH) of the invention compared to the wild-type ADH counterpart has a lower clearance value within the blood and/or serum. In another embodiment, long-acting alcohol dehydrogenase (ADH) of the invention compared to the wild-type ADH counterpart has a lower elimination rate value within the blood and/or serum. In another embodiment, long-acting alcohol dehydrogenase (ADH) of the invention compared to the wild-type ADH counterpart has a higher t½ measure within the blood and/or serum. In another embodiment, long-acting alcohol dehydrogenase (ADH) of the invention compared to the wild-type ADH counterpart has a higher Cₘₐₓ and/or C_{Z} value within the blood and/or serum. In one embodiment, phrases: “enhanced longevity” and “long-acting” are used interchangeably.

In another embodiment, an ADH of the present invention is a chimeric protein. In another embodiment, an ADH of the present invention is a recombinant protein. In another embodiment, the present invention provides a chimeric and/or a recombinant ADH protein and DNA molecules encoding a chimeric and/or a recombinant ADH protein.

In another embodiment, an ADH of the present invention acts on primary or secondary alcohols or hemi-acetals. In another embodiment, an ADH of the present invention oxidizes EtOH much better than methanol. In another embodiment, an ADH of the present invention acts on cyclic secondary alcohols. In another embodiment, an ADH of the present invention facilitates the interconversion between alcohols and aldehydes or ketones with the reduction of nicotinamide adenine dinucleotide (NAD+ to NADH). In another embodiment, an ADH of the present invention breaks down alcohols. In another embodiment, an ADH of the present invention breaks down alcohols that otherwise are toxic.

In another embodiment, an ADH of the present invention is attached to cyclodextrin.

In another embodiment, an ADH of the present invention is covalently attached to poly(ethylene glycol) (PEG). In another embodiment, an ADH of the present invention is covalently attached to polysialic acid (PSA). In another embodiment, PEG is linear. In another embodiment, PEG is branched.

In another embodiment, an ADH of the present invention is PEGylated. In another embodiment, an ADH of the present invention is modified with a detachable PEG molecule (reversible PEGylation). In another embodiment, an ADH of the present invention is a chimeric protein and/or a recombinant protein comprising at least one repeat of the artificial repetitive sequence PSTAD.

In another embodiment, an ADH of the present invention is a chimeric protein and/or a recombinant protein fused to serum albumin such as HAS. In another embodiment, an ADH of the present invention is a chimeric protein and/or a recombinant protein fused to a fragment of serum albumin. In another embodiment, an ADH of the present invention is a chimeric protein and/or a recombinant protein comprising at least one repeat of the C-terminal peptide (CTP) of a chorionic gonadotropin. In another embodiment, an ADH of the present invention is a chimeric protein and/or a recombinant protein fused to the constant fragment (Fc) domain of an immunoglobulin (Ig) G. In another embodiment, an ADH of the present invention is a chimeric protein and/or a recombinant protein fused to XTEN.

In another embodiment, an ADH of the present invention is a chimeric protein and/or a recombinant protein comprising at least one repeat of the C-terminal peptide (CTP) of a mammal chorionic gonadotropin. In another embodiment, an ADH of the present invention is a chimeric protein and/or a recombinant protein comprising at least one repeat of the C-terminal peptide (CTP) of human chorionic gonadotropin. In another embodiment, the CTP peptide is a CTP as described in U.S. Pat. No. 5,712,122 which is hereby incorporated by reference in its entirety.

In another embodiment, the CTP peptide is a variant of chorionic gonadotrophin CTP which differs from the native CTP by 1-5 conservative amino acid substitutions as described in U.S. Pat. No. 5,712,122. In another embodiment, the CTP peptide is a variant of chorionic gonadotrophin CTP which differs from the native CTP by 1 conservative amino acid substitution. In another embodiment, the CTP peptide is a variant of chorionic gonadotrophin CTP which differs from the native CTP by 2 conservative amino acid substitutions. In another embodiment, the CTP peptide is a variant of chorionic gonadotrophin CTP which differs from the native CTP by 3 conservative amino acid substitutions. In another embodiment, the CTP peptide is a variant of chorionic gonadotrophin CTP which differs from the native CTP by 4 conservative amino acid substitutions. In another embodiment, the CTP peptide is a variant of chorionic gonadotrophin CTP which differs from the native CTP by 5 conservative amino acid substitutions. In another embodiment, the CTP peptide amino acid sequence of the present invention is at least 70% homologous to the native CTP amino acid sequence or a peptide thereof. In another embodiment, the CTP peptide amino acid sequence of the present invention is at least 80% homologous to the native CTP amino acid sequence or a peptide thereof. In another embodiment, the CTP peptide amino acid sequence of the present invention is at least 90% homologous to the native CTP amino acid sequence or a peptide thereof. In another embodiment, the CTP peptide amino acid sequence of the present invention is at least 95% homologous to the native CTP amino acid sequence or a peptide thereof

In another embodiment, an ADH of the present invention is a chimeric protein and/or a recombinant protein comprising at least one additional glycosylation site or amino acid residue, compared to the wild-type ADH. In another embodiment, an ADH of the present invention is hyperglycosylated, compared to the wild-type ADH. In another embodiment, an ADH of the present invention further comprises N-linked oligosaccharides sequence: Asn-Xxx-Ser/Thr where Xxx is anything but proline. In another embodiment, an ADH of the present invention further comprises at least one serine and/or threonine residue. In another embodiment, an ADH of the present invention further comprises at least one inert peptide repeat polymer (hybrid of the PEG conjugation approach and fusion to the naturally long-half-life proteins IgG Fc, albumin, or transferrin).

In another embodiment, an ADH of the present invention comprises a higher glycosylation degree compared to the wild-type ADH. In another embodiment, an ADH of the present invention comprises a higher glycan size compared to the wild-type ADH. In another embodiment, an ADH of the present invention comprises elevated number of charged terminal glycans (e.g., sialic acid) compared to the wild-type ADH. In another embodiment, an ADH of the present invention is modified by conjugation to a polyamino acid polymer (e.g. polyglutamic acid (PGA), N-(2-hydroxypropyl) methacrylamide copolymer (HPMA), and hybrid modified PEG polymers). In another embodiment, an ADH of the present invention is modified by attachment to a polymer comprising glutamic acid and vitamin E (Medusa® polymer). In another embodiment, an ADH of the present invention is modified with a hybrid PEG polymer such as but not limited to PGC™. In another embodiment, an ADH of the present invention is modified with starch. In another embodiment, an ADH of the present invention is modified with Hydroxyethyl starch (HES). In another embodiment, an ADH of the present invention is modified with XTEN ranging from 36 to 288 amino acid residues in length (also known as recombinant PEG or “rPEG”). In another embodiment, an ADH of the present invention is modified with a homo-amino acid polymer (HAP). In another embodiment, an ADH of the present invention is modified with a proline-alanine-serine polymer (PAS). In another embodiment, PAS is a polymer of 50-400 repeats. In another embodiment, an ADH of the present invention is modified with an elastin-like peptide (ELP). In another embodiment, an ADH of the present invention is modified with a gelatin-like protein (GLK) polymer. In another embodiment, gelatin-like protein (GLK) polymer, constant fragment (Fc) domain of an immunoglobulin (Ig) G, CTP repeat, glycosylating amino acid residue, hyperglycosylated amino acid sequence, N-linked oligosaccharides sequence, Asn-Xxx-Ser/Thr where Xxx is anything but proline, serine and/or threonine residue, inert peptide repeat polymer, albumin, transferrin, charged terminal glycan, polyamino acid polymer, PEG polymer, hybrid modified PEG polymers, HPMA, glutamic acid and vitamin E, Medusa® polymer, HES, XTEN, rPEG, HAP, PAS, or any combination thereof is/are referred to as “long-acting molecule”. In another embodiment, “long-acting molecule” is any amino acid, amino acid sequence or a molecule capable of extending the t½ of a peptide or a protein. In another embodiment, “long-acting molecule” is any amino acid, amino acid sequence or a molecule capable of extending the t½ of ADH/KRED. In another embodiment, “long-acting molecule” is any amino acid, amino acid sequence or a molecule capable of enhancing Cₘₐₓ of ADH/KRED. In another embodiment, “long-acting molecule” is any amino acid, amino acid sequence or a molecule capable of extending the AUC of ADH/KRED. In another embodiment, “long-acting molecule” is any amino acid, amino acid sequence or a molecule capable of extending Km of ADH/KRED. In another embodiment, “long-acting molecule” is any amino acid, amino acid sequence or a molecule capable of extending/enhancing Vₘₐₓ of ADH/KRED.

In another embodiment, an ADH of the present invention is a fusion protein (chimera and/or recombinant) comprising ADH coupled to a naturally long-half-life protein or protein domain such as but not limited to Fc fusion, transferrin, CTP, albumin, or any combination thereof. In another embodiment, the ADH part of the fused protein is the biologically active portion of the chimera. In another embodiment, the naturally long-half-life protein or a domain thereof is coupled to: the amino terminus of ADH, the carboxy terminus of ADH, or both termini.

In another embodiment, any modification as described herein includes the addition of at least one amino acid residue to the coding sequence of ADH. In another embodiment, any modification as described herein includes the addition of at least two amino acid residues to the coding sequence of ADH. In another embodiment, any modification as described herein includes the addition of at least 3 amino acid residues to the coding sequence of ADH. In another embodiment, any modification as described herein includes the addition of at least 4 amino acid residues to the coding sequence of ADH. In another embodiment, any modification as described herein includes the addition of at least 5 amino acid residues to the coding sequence of ADH. In another embodiment, any modification as described herein includes the addition of at least 6 amino acid residues to the coding sequence of ADH. In another embodiment, any modification as described herein includes the addition of at least 7 amino acid residues to the coding sequence of ADH. In another embodiment, any modification as described herein includes the addition of at least 8 amino acid residues to the coding sequence of ADH. In another embodiment, any modification as described herein includes the addition of at least 9 amino acid residues to the coding sequence of ADH. In another embodiment, any modification as described herein includes the addition of at least 10 amino acid residues to the coding sequence of ADH. In another embodiment, ADH includes KRED proteins. In another embodiment, an ADH and/or KRED of the invention is disclosed in U.S. Pat. No. 7,833,767 which is hereby incorporated by reference in its entirety in another embodiment, an ADH and/or KRED of the invention is disclosed in International Publication No.

In another embodiment, an ADH and/or KRED of the invention is disclosed in International Publication No. In another embodiment, any mutant of ADH and/or KRED is also encompassed by the terms “ADH”, “KRED” and/or “ADH/KRED”. In another embodiment, ADH/KRED is also a biologically active fragment of ADH/KRED. In another embodiment, a “fragment” is an ADH/KRED having a deletion of 1 to 10 amino acid residues. In another embodiment, a “fragment” is meant that the ADH/KRED has a deletion of 2 to 8 amino acid residues. In another embodiment, a “fragment” is meant that the ADH/KRED has a deletion of 1 to 15 amino acid residues from the carboxy terminus. In another embodiment, a “fragment” is meant that the ADH/KRED has a deletion of 1 to 15 amino acid residues from the amino terminus. In another embodiment, a “fragment” is meant that the ADH/KRED has a deletion of 1 to 15 amino acid residues from both the carboxy terminus and the amino terminus. In another embodiment, a “fragment” is meant that the ADH/KRED has a deletion of 1 to 10 amino acid residues from the carboxy terminus, the amino terminus, or both. In another embodiment, a “fragment” is meant that the ADH/KRED has a deletion of 2 to 8 amino acid residues from the carboxy terminus, the amino terminus, or both.

In another embodiment, an ADH/KRED as described herein is a KRED purchased from Codexis. In another embodiment, an ADH/KRED as described herein is described in Codexis' Codex Screening Kit. In another embodiment, an ADH/KRED as described herein is described in Codexis' Codex Screening Kit (Screening Protocol version date 2013-10-04).

In another embodiment, “modification” as described herein is rendering an ADH long-acting ADH (compared to the wild-type ADH counterpart). In another embodiment, long-acting is in-vivo or ex-vivo long-acting. In another embodiment, long-acting is blood long-acting. In another embodiment, long-acting is plasma long-acting. In another embodiment, long-acting is bodily fluid long-acting. In another embodiment, long-acting is intestine long-acting. In another embodiment, long-acting is stomach long-acting.

In another embodiment, any modification as described herein includes the addition of 1 to 60 amino acid residues to the coding sequence of ADH. In another embodiment, any modification as described herein includes the addition of 1 to 25 amino acid residues to the coding sequence of ADH. In another embodiment, any modification as described herein includes the addition of 3 to 25 amino acid residues to the coding sequence of ADH. In another embodiment, any modification as described herein includes the addition of 5 to 30 amino acid residues to the coding sequence of ADH. In another embodiment, any modification as described herein includes the addition of 12 to 60 amino acid residues to the coding sequence of ADH. In another embodiment, any modification as described herein includes the addition of 6 to 40 amino acid residues to the coding sequence of ADH.

In one embodiment, the present invention describes long-acting ADH/KRED and methods of producing and using same. In another embodiment, long-acting ADH/KRED comprise one or more of the molecules than confer long-acting activity of ADH/KRED. In another embodiment, long-acting ADH/KRED or a chimera of the invention comprises one or more of the molecules than confer long-acting activity of ADH/KRED.

In one embodiment, the present invention describes long-acting ADH/KRED (fusion/recombinant/chimera) comprising at least one long-acting molecule. In one embodiment, the present invention describes long-acting ADH/KRED (fusion/recombinant/chimera) comprising at least two long-acting molecules. In one embodiment, the present invention describes long-acting ADH/KRED (fusion/recombinant/chimera) comprising at least three long-acting molecules. In one embodiment, the present invention describes long-acting ADH/KRED (fusion/recombinant/chimera) comprising at least four long-acting molecules. In one embodiment, the present invention describes long-acting ADH/KRED (fusion/recombinant/chimera) comprising at least five long-acting molecules. In one embodiment, the present invention describes long-acting ADH/KRED (fusion/recombinant/chimera) comprising at least six long-acting molecules.

In one embodiment, the present invention describes long-acting ADH/KRED (fusion/recombinant/chimera) comprising 1 to 20 long-acting molecules. In one embodiment, the present invention describes long-acting ADH/KRED (fusion/recombinant/chimera) comprising 1 to 5 long-acting molecules. In one embodiment, the present invention describes long-acting ADH/KRED (fusion/recombinant/chimera) comprising 2 to 8 long-acting molecules. In one embodiment, the present invention describes long-acting ADH/KRED (fusion/recombinant/chimera) comprising 2 to 5 long-acting molecules. In one embodiment, the present invention describes long-acting ADH/KRED (fusion/recombinant/chimera) comprising 3 to 7 long-acting molecules. In one embodiment, the present invention describes long-acting ADH/KRED (fusion/recombinant/chimera) comprising 5 to 15 long-acting molecules.

In another embodiment, a plurality of long-acting molecules means a plurality of one long-acting molecule such as but not limited to a plurality of PEG molecules. In another embodiment, a plurality of long-acting molecules means a combination of different long-acting molecules such as but not limited to a PEG molecule and a CTP molecule.

In another embodiment, a long-acting ADH/KRED comprises at least one long-acting molecule attached to ADH/KRED, wherein at least one long-acting molecule is attached to the amino terminus of ADH/KRED. In another embodiment, a long-acting ADH/KRED comprises at least one long-acting molecule attached to ADH/KRED, wherein at least one long-acting molecule is attached to the carboxy terminus of ADH/KRED. In another embodiment, a long-acting ADH/KRED comprises at least two long-acting molecules attached to ADH/KRED, wherein a first long-acting molecules of the at least two long-acting molecules is attached to the amino terminus of ADH/KRED and a second long-acting molecule of the at least two long-acting molecules is attached to the carboxy terminus of the ADH/KRED.

In another embodiment, a long-acting molecule is attached to ADH/KRED via a linker. In another embodiment, the linker which connects a long-acting molecule to ADH/KRED is a covalent bond. In another embodiment, the linker which connects a long-acting molecule to the ADH/KRED is a peptide bond. In another embodiment, the linker which connects a long-acting molecule to ADH/KRED is a substituted peptide bond.

In another embodiment, a complexing molecule is attached to ADH/KRED include, but are not limited, cyclodextrin.

The phrase “ADH/KRED”, “ADH or “KRED”” refers, in another embodiment, to any polypeptide having ADH biological activity. In another embodiment, ADH/KRED is glycosylated. In another embodiment, ADH/KRED is non-glycosylated. In another embodiment, a long-acting molecule is fused to an ADH/KRED.

In some embodiments, placing a long-acting molecule at both the amino terminal end of an ADH/KRED and at the carboxy terminal end of an ADH/KRED provide enhanced protection against degradation of ADH/KRED. In some embodiments, placing a long-acting molecule at both the amino terminal end of an ADH/KRED and at the carboxy terminal end of an ADH/KRED provide extended half-life of the attached ADH/KRED.

In some embodiments, a long-acting molecule at the amino terminal end of an ADH/KRED, a long-acting molecule at the carboxy terminal end of ADH/KRED, and at least one additional long-acting molecule attached in tandem to the long-acting molecule at the carboxy terminus provide enhanced protection against degradation of ADH/KRED. In some embodiments, a long-acting molecule at the amino terminal end of an ADH/KRED, a long-acting molecule at the carboxy terminal end of ADH/KRED, and at least one additional long-acting molecule sequence attached in tandem to the ADH/KRED sequence at the carboxy terminus provide extended half-life to the attached ADH/KRED.

In some embodiments, a long-acting molecule at the amino terminal end of an ADH/KRED, a long-acting molecule at the carboxy terminal end of ADH/KRED, and at least one additional long-acting molecule attached in tandem to the long-acting molecule at the carboxy terminus provide enhanced activity of the attached ADH/KRED.

In some embodiments, a long-acting molecule at the amino terminal end of an ADH/KRED, a long-acting molecule at the carboxy terminal end of ADH/KRED, and at least one additional long-acting molecule attached in tandem to the long-acting molecule at the amino terminus provide enhanced protection against degradation of the attached ADH/KRED. In some embodiments, a long-acting molecule at the amino terminal end of an ADH/KRED, a long-acting molecule at the carboxy terminal end of ADH/KRED, and at least one additional long-acting molecule attached in tandem to the long-acting molecule at the amino terminus provide extended half-life of the attached ADH/KRED. In some embodiments, a long-acting molecule at the amino terminal end of an ADH/KRED, a long-acting molecule at the carboxy terminal end of ADH/KRED, and at least one additional long-acting molecule attached in tandem to the long-acting molecule at the amino terminus provide enhanced activity the attached ADH/KRED.

In one embodiment, ADH/KRED of the present invention also refers to homologues. In one embodiment, ADH/KRED amino acid sequence of the present invention is at least 50% homologous to ADH/KRED listed on NCBI and/or determined using BlastP software of the National Center of Biotechnology Information (NCBI) using default parameters. In one embodiment, ADH/KRED sequence of the present invention is at least 60% homologous to ADH/KRED listed on NCBI and/or determined using BlastP software of the National Center of Biotechnology Information (NCBI) using default parameters. In one embodiment, interferon amino acid sequence of the present invention is at least 70% homologous to ADH/KRED listed on NCBI and/or determined using BlastP software of the National Center of Biotechnology Information (NCBI) using default parameters. In one embodiment, interferon amino acid sequence of the present invention is at least 80% homologous to ADH/KRED listed on NCBI and/or determined using BlastP software of the National Center of Biotechnology Information (NCBI) using default parameters. In one embodiment, interferon amino acid sequence of the present invention is at least 90% homologous to ADH/KRED listed on NCBI and/or determined using BlastP software of the National Center of Biotechnology Information (NCBI) using default parameters. In one embodiment, interferon amino acid sequence of the present invention is at least 95% homologous to ADH/KRED listed on NCBI and/or determined using BlastP software of the National Center of Biotechnology Information (NCBI) using default parameters. In some embodiments, homology according to the present invention also encompasses deletions, insertions, or substitution variants, including an amino acid substitution, thereof and biologically active polypeptide fragments thereof.

In some embodiments, an ADH/KRED is a known ADH/KRED having its sequence disclosed in a gene bank. In some embodiments, an ADH/KRED is a homologue of a known ADH/KRED which retains in-vivo and/or ex-vivo ADH activity. In another embodiment, a “homologue” or “homology” according to the present invention also encompasses deletions, insertions, or substitution variants, including an amino acid substitution, thereof and biologically active polypeptide fragments thereof.

In another embodiment, the methods of the present invention provide a nucleic acid sequence encoding an ADH/KRED (the protein) having additionally at least one long-acting molecule on the N-terminus and/or at least one long-acting molecule on the C-terminus for reducing blood alcohol level or blood alcohol concentration. In another embodiment, the methods of the present invention provide a nucleic acid sequence encoding an ADH/KRED having additionally one long-acting molecule on the N-terminus and/or two long-acting molecules on the C-terminus for reducing blood alcohol level or blood alcohol concentration.

In another embodiment, the invention further provides a composition and/or a pharmaceutical composition, comprising 10 mg to 100 g of long-acting ADH/KRED. In one embodiment, the present invention provides a composition and/or a pharmaceutical composition, comprising 10 mg to 10 g of long-acting ADH/KRED. In another embodiment, long-acting ADH/KRED is a Codexis KRED fused or linked to a long-acting molecule. In another embodiment, Codexis KRED is KRED-P1-A04, KRED-P1-A12, KRED-P1-B05, KRED-P1-B10, or any combination thereof. In one embodiment, the present invention provides a composition, comprising KRED and/or a long-acting ADH/KRED and acceptable carrier or diluent. In another embodiment, the pharmaceutical composition is an injectable pharmaceutical composition.

In another embodiment, the pharmaceutical composition comprises 10 mg to 8 g of KRED and/or long-acting ADH/KRED. In another embodiment, the pharmaceutical composition comprises 10 mg to 5 g of KRED and/or long-acting ADH/KRED. In another embodiment, the pharmaceutical composition comprises 10 mg to 1 g of KRED and/or long-acting ADH/KRED. In another embodiment, the pharmaceutical composition comprises 10 mg to 100 mg of KRED and/or long-acting ADH/KRED. In another embodiment, the pharmaceutical composition comprises 100 mg to 0.5 g of KRED and/or long-acting ADH/KRED. In another embodiment, the pharmaceutical composition comprises 1 mg to 100 mg of KRED and/or long-acting ADH/KRED. In another embodiment, the pharmaceutical composition comprises 5 mg to 300 mg of KRED and/or long-acting ADH/KRED. In another embodiment, the pharmaceutical composition comprises 500 mg to 30 g of KRED and/or long-acting ADH/KRED. In another embodiment, the pharmaceutical composition comprises 1 g to 30 g of KRED. In another embodiment, the pharmaceutical composition comprises 5 m to 50 g of KRED and/or long-acting ADH/KRED.

In another embodiment, provided herein a method for lowering blood alcohol in a subject in need thereof, comprising administering to said subject a composition comprising an effective amount of KRED and/or long-acting ADH/KRED, thereby lowering blood alcohol in a subject in need thereof. In another embodiment, an effective amount is 1 to 5000 mg/kg (body weight). In another embodiment, an effective amount is 0.2 to 5 mg/kg (body weight). In another embodiment, an effective amount is 0.5 to 100 mg/kg (body weight).

In another embodiment, provided herein a method for reducing risk associated with high blood alcohol concentration and/or chronic elevated blood alcohol concentration, comprising administering to a subject consuming alcohol, a composition comprising an effective amount of KRED and/or long-acting ADH/KRED. In another embodiment, a risk associated with high blood alcohol concentration and/or chronic elevated blood alcohol concentration is: neuronal damage, liver damage, brain damage, kidney damage, gastrointestinal damage, endocrine damage, diabetes, a cardiovascular disease, blindness, stroke, a metabolic disease, or any combination thereof.

In another embodiment, an effective amount is 1 to 50 mg/kg (body weight). In another embodiment, an effective amount is 20 to 300 mg/kg (body weight). In another embodiment, an effective amount is 100 to 1000 mg/kg (body weight). In another embodiment, an effective amount is 10 to 90 mg/kg (body weight). In another embodiment, an effective amount is 50 to 250 mg/kg (body weight). In another embodiment, an effective amount is 100 to 500 mg/kg (body weight).

In another embodiment, a subject in need thereof has a blood ethanol concentration of above 0.0001% by blood volume. In another embodiment, a subject in need thereof has a blood ethanol concentration of above 0.001% by blood volume. In another embodiment, a subject in need thereof has a blood ethanol concentration of above 0.1% by blood volume. In another embodiment, a subject in need thereof has a blood ethanol concentration of above 0.5% by blood volume.

In another embodiment, lowering blood alcohol level is alcohol detoxification. In another embodiment, lowering blood alcohol level (concentration) is reducing the neurological side effects of alcohol. In another embodiment, lowering blood alcohol level is reducing the damages exerted to tissues by alcohol. In another embodiment, lowering blood alcohol level is reducing the damages exerted to the liver by alcohol. In another embodiment, lowering blood alcohol level is reducing the risks associated with alcohol consumption.

In another embodiment, provided herein a method for preventing a symptom or a risk arising from alcohol consumption in a subject in need thereof, comprising administering to a subject a composition comprising an effective amount of KRED and/or a long-acting ADH/KRED, thereby preventing a symptom or a risk arising from alcohol consumption in a subject in need thereof. In another embodiment, a subject in need is a subject about to consume alcohol. In another embodiment, a subject about to consume alcohol is about to consume alcohol within 2 to 1000 minutes. In another embodiment, a subject about to consume alcohol is about to consume alcohol within 2 to 500 minutes. In another embodiment, a subject about to consume alcohol is about to consume alcohol within 20 to 700 minutes. In another embodiment, a subject about to consume alcohol is about to consume alcohol within 60 to 600 minutes. In another embodiment, a subject about to consume alcohol is about to consume alcohol within 2 to 60 minutes. In another embodiment, a subject about to consume alcohol is about to consume alcohol within 2 to 30 minutes. In another embodiment, a subject about to consume alcohol is about to consume alcohol within 5 to 60 minutes. In another embodiment, a subject about to consume alcohol is about to consume alcohol within 20 to 120 minutes. In another embodiment, a subject about to consume alcohol is about to consume alcohol within 10 to 90 minutes.

In another embodiment, provided herein a method for treating a subject afflicted with alcoholism, comprising administering to said subject a composition comprising an effective amount of KRED and/or a long-acting ADH/KRED. In another embodiment, provided herein a method for reducing blood alcohol level and/or reducing symptoms and risks associated with alcohol, comprising administering a composition as described herein before alcohol consumption, after alcohol consumption, during or both before and after alcohol consumption, or any combination thereof.

In another embodiment, a KRED of the invention belongs to the ketoreductase (KRED) or carbonyl reductase class (EC 1. 1. 1. 184). In another embodiment, a KRED and/or a long-acting ADH/KRED of the invention oxidizes an alcohol substrate to the corresponding ketone/aldehyde product in the presence of a co-factor such as nicotinamide adenine dinucleotide (NADH) or reduced nicotinamide adenine dinucleotide phosphate (NADPH), and nicotinamide adenine dinucleotide (NAD) or nicotinamide adenine dinucleotide phosphate (NADP).

In another embodiment, a KRED of the invention is disclosed in U.S. Pat. No. 7,833,767 which is hereby incorporated by reference in its entirety. In another embodiment, a KRED of the invention comprises the following amino acid sequence: MAKNFSNVEY PAPPPAHTKNESLQVLDLFKLNGKVASITGSSSGIGYALAEAFAQVGADVAIWYNSHDAT GKAEALAKKYGVKVKAYKANVSSSDAVKQTIEQQIKDFGHLDIVVANAGIPWTKGAYID QDDDKHFDQVVDVDLKGVGYVAKHAGRHFRERFEKEGKKGALVFTASMSGHIVNVPQ FQATYNAAKAGVRHFAKSLAVEFAPFARVNSVSPGYINTEISDFVPQETQNKWWSLVPL GRGGETAELVGAYLFLASDAGSYATGTDIIVDGGYTLP (SEQ ID NO: 1). In another embodiment, a KRED of the invention has enhanced KRED activity relative to a KRED of SEQ ID NO: 1. In another embodiment, a KRED of the invention differs from SEQ ID NO: 1 by 1-25 amino acid residues. In another embodiment, a KRED of the invention differs from SEQ ID NO: 1 by 1-20 amino acid residues. 1. In another embodiment, a KRED of the invention differs from SEQ ID NO: 1 by 5-15 amino acid residues. In another embodiment, a KRED of the invention differs from SEQ ID NO: 1 by 2-8 amino acid residues. In another embodiment, a KRED of the invention comprises a mutated form of SEQ ID NO: 1. In another embodiment, ADH/KRED as described herein comprises KRED as described herein.

In another embodiment, a KRED of the invention comprises SEQ ID Nos: 506, 520, 526, 536, and 538 of International Publication No. (PCT): WO2005017135 which is hereby incorporated by reference in its entirety. In another embodiment, KRED and/or a long-acting ADH/KRED (PCT): WO2014174505 which is hereby incorporated by reference in its entirety.

In another embodiment, a KRED and/or a long-acting ADH/KRED of the invention is or comprises a fragment of SEQ ID NO: 1. In another embodiment, a KRED and/or a long-acting ADH/KRED of the invention is or comprises a fragment of SEQ ID NO: 1 having from 1.2 to about 100 times the ADH/KRED activity of the unmodified ADH/KRED, when measured as the lysate. In another embodiment, a long-acting KRED and/or ADH/KRED has at least comparable activity to the wild-type KRED of SEQ ID NO: 1. In another embodiment, a “fragment” is meant that the polypeptide has a deletion of 1 to 15 amino acid residues. In another embodiment, a “fragment” is meant that the polypeptide has a deletion of 1 to 10 amino acid residues. In another embodiment, a “fragment” is meant that the polypeptide has a deletion of 2 to 8 amino acid residues. In another embodiment, a “fragment” is meant that the polypeptide has a deletion of 1 to 15 amino acid residues from the carboxy terminus. In another embodiment, a “fragment” is meant that the polypeptide has a deletion of 1 to 15 amino acid residues from the amino terminus. In another embodiment, a “fragment” is meant that the polypeptide has a deletion of 1 to 15 amino acid residues from both the carboxy terminus and the amino terminus. In another embodiment, a “fragment” is meant that the polypeptide has a deletion of 1 to 10 amino acid residues from the carboxy terminus, the amino terminus, or both. In another embodiment, a “fragment” is meant that the polypeptide has a deletion of 2 to 8 amino acid residues from the carboxy terminus, the amino terminus, or both.

In another embodiment, a mutated form of SEQ ID NO: 1 comprises the following amino acid residue replacement or replacements: A2V; K3E; F5L or C; N7K; E9G or K; A12V; P13L; P14A; A16G or V; T18A; K19I; N20D or S; E21K; S22N or T; Q24H or R; V25A; N32S or D; A36T; S41G; S42N; I45L; A48T; V56A; V60I; Y64H; N65K, D, Y or S; S66G or R; H67L or Q; D68G or N; G71D; E74K or G; K78R; K79R; K85R; A86V; N9OD; S93Nor C; D95N, G, V, Y or E; K98R; Q99L, R, or H; T100A; I101V; Q103R; 1105V or T; K106R or Q; H110Y, C or R; V114A; A116G; I120V; K124R; D129G or N; D131G or V; D132N; K134M, V, E or R; D137N or G; Q138L; V140I; D143N; L144F; K145R; V147A; V150A; H153Y or Q; H157Y; F158L or Y; R159K; E160G or V; F162Y or S; E163G or K; E165D, G or K; K1671 or R; A170S; V172I; F173C; M177V or T; H180Y; V184I; T190A; A193V; A194V; F201L; K203R; F209Y; V218I; N224S; E226K, G or D; S228T; D229A; V231I or A; Q233K or R; E234G or D; T235K or A; N237Y; K238R or E; T251A; V255A; F260L; A262V; T272A; I274L; I275L or V; P283R, or any combination thereof.

In another embodiment, a KRED as described herein is a KRED purchased from Codexis. In another embodiment, a KRED as described herein is described in Codexis' Codex Screening Kit. In another embodiment, a KRED as described herein is described in Codexis' Codex Screening Kit (Screening Protocol version date 2013-10-04).

In another embodiment, a KRED and/or a long-acting ADH/KRED as described herein has from 1.2 to about 10 times or from 1.2 to about 100 times the ADH/KRED activity of the unmodified ADH/KRED or the unmodified KRED activity of the polypeptide of SEQ ID NO: 1, when measured as the lysate. In another embodiment, a KRED as described herein has from 1.2 to about 10 times the KRED activity of the polypeptide of SEQ ID NO: 1, when measured as the lysate. In another embodiment, a long-acting ADH/KRED or KRED as described herein has from 2 to about 50 times the KRED and/or ADH/KRED activity of the unmodified KRED and/or ADH/KRED polypeptide of SEQ ID NO: 1, when measured as the lysate. In another embodiment, a long-acting ADH/KRED and/or KRED as described herein has from 4 to about 30 times the ADH/KRED and/or KRED activity of the KRED, unmodified KRED and/or ADH/KRED polypeptide of SEQ ID NO: 1, when measured as the lysate.

In another embodiment, a long-acting ADH/KRED as described herein has enhanced protection against degradation compared to a protein comprising SEQ ID NO: 1. In another embodiment, a KRED and/or long-acting ADH/KRED as described herein has enhanced protection against degradation under physiological conditions compared to the unmodified ADH/KRED. In another embodiment, any KRED comprising polypeptide as described herein has enhanced thermostability compared to the unmodified ADH/KRED or to a protein comprising SEQ ID NO: 1. In another embodiment, KRED and/or a long-acting ADH/KRED as described herein has enhanced thermostability under physiological conditions compared to the unmodified ADH/KRED and/or a protein comprising SEQ ID NO: 1.

In another embodiment, KRED and/or a long-acting ADH/KRED of the invention as described herein retains at least 15% of the initial (pre-incubation) KRED and/or ADH/KRED activity after its administration for 0.2-24 hours. In another embodiment, a long-acting ADH/KRED of the invention or the KRED of the invention as described herein retains at least 15% of the initial (pre-incubation) KRED and/or ADH/KRED activity after its administration for 0.2-2 hours. In another embodiment, KRED and/or a long-acting ADH/KRED of the invention as described herein retains at least 15% of the initial (pre-incubation) KRED and/or ADH/KRED activity after its administration for 0.2-4 hours. In another embodiment, KRED and/or a long-acting ADH/KRED of the invention as described herein retains at least 15% of the initial (pre-incubation) KRED and/or ADH/KRED activity after its administration for 1-7 hours.

In another embodiment, KRED and/or a long-acting ADH/KRED of the invention as described herein retains at least 15% of the initial (pre-incubation) KRED and/or ADH/KRED activity after its administration for 0.5-70 hours. In another embodiment, KRED and/or a long-acting ADH/KRED of the invention as described herein retains at least 15% of the initial (pre-incubation) KRED and/or ADH/KRED activity after its administration for 1-50 hours. In another embodiment, KRED and/or a long-acting ADH/KRED of the invention as described herein retains at least 15% of the initial (pre-incubation) KRED and/or ADH/KRED activity after its administration for 0.5-10 hours. In another embodiment, administration refers to administration of KRED and/or ADH/KRED into a physiological environment. In another embodiment, administration refers to KRED and/or ADH/KRED within blood. In another embodiment, administration refers to KRED and/or ADH/KRED within serum.

In another embodiment, at least 15% is 15% to 75%. In another embodiment, at least 15% is 15% to 50%. In another embodiment, at least 15% is 15% to 30%. In another embodiment, 0.5-10 hours is 0.5-4 hours. In another embodiment, 0.5-10 hours is 0.5-2 hours. In another embodiment, 0.5-10 hours is 0.5-4 hours. In another embodiment, 0.5-10 hours is 0.5-2.5 hours.

In another embodiment, KRED and/or an ADH/KRED as described herein has enhanced protection against degradation under standard protein storage conditions compared to unmodified ADH/KRED and/or a protein comprising SEQ ID NO: 1. In another embodiment, KRED and/or an ADH/KRED as described herein has from 1.2 to about 100 times the: serum half-life, circulatory half-life, AUC, CL, Ke, t½, Cmax, Tmax, Vdz, or any combination thereof compared to unmodified ADH/KRED and/or a protein comprising SEQ ID NO: 1.

In another embodiment, a DNA sequence encoding KRED and/or ADH/KRED of the invention is used in the process of manufacturing KRED and/or ADH/KRED as described herein. In another embodiment, a DNA sequence encoding KRED and/or ADH/KRED of the invention is used in the process of manufacturing a mutated KRED and/or ADH/KRED as described herein. In another embodiment, a DNA sequence encoding KRED and/or ADH/KRED is a vector or a plasmid.

In some embodiments, KRED and/or ADH/KRED is a polypeptide. In one embodiment, “polypeptide” as used herein encompasses native polypeptides (either degradation products, synthetically synthesized polypeptides or recombinant polypeptides) and peptidomimetics (typically, synthetically synthesized polypeptides), as well as peptoids and semipeptoids which are polypeptide analogs, which have, in some embodiments, modifications rendering the polypeptides even more stable while in a body or more capable of penetrating into cells. In one embodiment, “polypeptide” as used herein is KRED or ADH/KRED. In one embodiment, ADH/KRED is a long-acting ADH/KRED.

In some embodiments, modifications include, but are not limited to N terminus modification, C terminus modification, polypeptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

In some embodiments, polypeptide bonds (—CO—NH—) within the polypeptide are substituted. In some embodiments, the polypeptide bonds are substituted by N-methylated bonds (—N(CH3)-CO—). In some embodiments, the polypeptide bonds are substituted by ester bonds (—C(R)H—C—O—O—C(R)—N—). In some embodiments, the polypeptide bonds are substituted by ketomethylen bonds (—CO—CH2-). In some embodiments, the polypeptide bonds are substituted by α-aza bonds (—NH—N(R)—CO—), wherein R is any alkyl, e.g., methyl, carba bonds (—CH2-NH—). In some embodiments, the polypeptide bonds are substituted by hydroxyethylene bonds (—CH(OH)—CH2-). In some embodiments, the polypeptide bonds are substituted by thioamide bonds (—CS—NH—). In some embodiments, the polypeptide bonds are substituted by olefinic double bonds (—CH═CH—). In some embodiments, the polypeptide bonds are substituted by retro amide bonds (—NH—CO—). In some embodiments, the polypeptide bonds are substituted by polypeptide derivatives (—N(R)—CH2-CO—), wherein R is the “normal” side chain, naturally presented on the carbon atom. In some embodiments, these modifications occur at any of the bonds along the polypeptide chain and even at several (2-3 bonds) at the same time.

In some embodiments, natural aromatic amino acids of the polypeptide such as Trp, Tyr and Phe, be substituted for synthetic non-natural acid such as Phenylglycine, TIC, naphthylelanine (Nol), ring-methylated derivatives of Phe, halogenated derivatives of Phe or o-methyl-Tyr. In some embodiments, the polypeptides of the present invention include one or more modified amino acid or one or more non-amino acid monomers (e.g. fatty acid, complex carbohydrates etc).

In one embodiment, “amino acid” or “amino acid” is understood to include the 20 naturally occurring amino acid; those amino acid often modified post-translationally in vivo, including, for example, hydroxyproline, phosphoserine and phosphothreonine; and other unusual amino acid including, but not limited to, 2-aminoadipic acid, hydroxylysine, isodesmosine, nor-valine, nor-leucine and ornithine. In one embodiment, “amino acid” includes both D- and L-amino acid.

In some embodiments, the polypeptides of the present invention are utilized in therapeutics which requires the polypeptides to be in a soluble form. In some embodiments, the polypeptides of the present invention include one or more non-natural or natural polar amino acid, including but not limited to serine and threonine which are capable of increasing polypeptide solubility due to their hydroxyl-containing side chain.

In some embodiments, the polypeptides of the present invention are utilized in a linear form, although it will be appreciated by one skilled in the art that in cases where cyclicization does not severely interfere with polypeptide characteristics, cyclic forms of the polypeptide can also be utilized.

In some embodiments, the polypeptides of present invention are biochemically synthesized such as by using standard solid phase techniques. In some embodiments, these biochemical methods include exclusive solid phase synthesis, partial solid phase synthesis, fragment condensation, or classical solution synthesis. In some embodiments, these methods are used when the polypeptide is relatively short (about 5-15 kDa) and/or when it cannot be produced by recombinant techniques (i.e., not encoded by a nucleic acid sequence) and therefore involves different chemistry.

In some embodiments, solid phase polypeptide synthesis procedures are well known to one skilled in the art and further described by John Morrow Stewart and Janis Dillaha Young, Solid Phase Polypeptide Syntheses (2nd Ed., Pierce Chemical Company, 1984). In some embodiments, synthetic polypeptides are purified by preparative high-performance liquid chromatography [Creighton T. (1983) Proteins, structures and molecular principles. WH Freeman and Co. N.Y.] and the composition of which can be confirmed via amino acid sequencing by methods known to one skilled in the art.

In some embodiments, recombinant protein techniques are used to generate the polypeptides of the present invention. In some embodiments, recombinant protein techniques are used for generation of relatively long polypeptides (e.g., longer than 18-25 amino acid). In some embodiments, recombinant protein techniques are used for the generation of large amounts of the polypeptide of the present invention. In some embodiments, recombinant techniques are described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al, (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

In one embodiment, a polypeptide of the present invention is synthesized using a polynucleotide encoding the polypeptide of the present invention. In some embodiments, the polynucleotide encoding a polypeptide of the present invention is ligated into an expression vector, comprising a transcriptional control of a cis-regulatory sequence (e.g., promoter sequence). In some embodiments, the cis-regulatory sequence is suitable for directing constitutive expression of the polypeptide of the present invention. In some embodiments, the cis-regulatory sequence is suitable for directing tissue specific expression of the polypeptide of the present invention. In some embodiments, the cis-regulatory sequence is suitable for directing inducible expression of the polypeptide of the present invention.

In some embodiments, polynucleotides which express the polypeptides of the present invention are as set forth in SEQ ID NOs: 20, 21, 44, 45 and 46.

In some embodiment, tissue-specific promoters suitable for use with the present invention include sequences which are functional in specific cell population, example include, but are not limited to promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166). Inducible promoters suitable for use with the present invention include for example the tetracycline-inducible promoter (Srour, M. A., et al., 2003. Thromb. Haemost. 90: 398-405).

In one embodiment, the phrase “a polynucleotide” refers to a single or double stranded nucleic acid sequence which be isolated and provided in the form of an RNA sequence, a complementary polynucleotide sequence (cDNA), a genomic polynucleotide sequence and/or a composite polynucleotide sequences (e.g., a combination of the above).

In one embodiment, “complementary polynucleotide sequence” refers to a sequence, which results from reverse transcription of messenger RNA using a reverse transcriptase or any other RNA dependent DNA polymerase. In one embodiment, the sequence can be subsequently amplified in vivo or in vitro using a DNA polymerase.

In one embodiment, “genomic polynucleotide sequence” refers to a sequence derived (isolated) from a chromosome and thus it represents a contiguous portion of a chromosome.

In one embodiment, “composite polynucleotide sequence” refers to a sequence, which is at least partially complementary and at least partially genomic. In one embodiment, a composite sequence can include some exonal sequences required to encode the polypeptide of the present invention, as well as some intronic sequences interposing there between. In one embodiment, the intronic sequences can be of any source, including of other genes, and typically will include conserved splicing signal sequences. In one embodiment, intronic sequences include cis acting expression regulatory elements.

In one embodiment, the polynucleotides of the present invention further comprise a signal sequence encoding a signal peptide for the secretion of the polypeptides of the present invention.

In one embodiment, following expression and secretion, the signal peptides are cleaved from the precursor proteins resulting in the mature proteins.

In some embodiments, polynucleotides of the present invention are prepared using PCR techniques as described in Example 1, or any other method or procedure known to one skilled in the art. In some embodiments, the procedure involves the legation of two different DNA sequences (See, for example, “Current Protocols in Molecular Biology”, eds. Ausubel et al., John Wiley & Sons, 1992).

In one embodiment, polynucleotides of the present invention are inserted into expression vectors (i.e., a nucleic acid construct) to enable expression of the recombinant polypeptide. In one embodiment, the expression vector of the present invention includes additional sequences which render this vector suitable for replication and integration in prokaryotes. In one embodiment, the expression vector of the present invention includes additional sequences which render this vector suitable for replication and integration in eukaryotes. In one embodiment, the expression vector of the present invention includes a shuttle vector which renders this vector suitable for replication and integration in both prokaryotes and eukaryotes. In some embodiments, cloning vectors comprise transcription and translation initiation sequences (e.g., promoters, enhances) and transcription and translation terminators (e.g., polyadenylation signals).

In one embodiment, a variety of prokaryotic or eukaryotic cells can be used as host-expression systems to express the polypeptides of the present invention. In some embodiments, these include, but are not limited to, microorganisms, such as bacteria transformed with a recombinant bacteriophage DNA, plasmid DNA or cosmid DNA expression vector containing the polypeptide coding sequence; yeast transformed with recombinant yeast expression vectors containing the polypeptide coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the polypeptide coding sequence.

In some embodiments, non-bacterial expression systems are used (e.g. mammalian expression systems such as CHO cells) to express the polypeptide of the present invention. In one embodiment, the expression vector used to express polynucleotides of the present invention in mammalian cells is pCI-DHFR vector comprising a CMV promoter and a neomycin resistance gene. Construction of the pCI-dhfr vector is described, according to one embodiment, in Example 1.

In some embodiments, in bacterial systems of the present invention, a number of expression vectors can be advantageously selected depending upon the use intended for the polypeptide expressed. In one embodiment, large quantities of polypeptide are desired. In one embodiment, vectors that direct the expression of high levels of the protein product, possibly as a fusion with a hydrophobic signal sequence, which directs the expressed product into the periplasm of the bacteria or the culture medium where the protein product is readily purified are desired. In one embodiment, certain fusion protein engineered with a specific cleavage site to aid in recovery of the polypeptide. In one embodiment, vectors adaptable to such manipulation include, but are not limited to, the pET series of E. coli expression vectors [Studier et al., Methods in Enzymol. 185:60-89 (1990)].

In one embodiment, yeast expression systems are used. In one embodiment, a number of vectors containing constitutive or inducible promoters can be used in yeast as disclosed in U.S. Pat. No. 5,932,447. In another embodiment, vectors which promote integration of foreign DNA sequences into the yeast chromosome are used.

In one embodiment, the expression vector of the present invention can further include additional polynucleotide sequences that allow, for example, the translation of several proteins from a single mRNA such as an internal ribosome entry site (IRES) and sequences for genomic integration of the promoter-chimeric polypeptide.

In some embodiments, mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors are useful for in vivo expression of the polypeptides of the present invention since they offer advantages such as lateral infection and targeting specificity. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

In one embodiment, various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In some embodiments, introduction of nucleic acid by viral infection offers several advantages over other methods such as lipofection and electroporation, since higher transfection efficiency can be obtained due to the infectious nature of viruses.

In one embodiment, it will be appreciated that the polypeptides of the present invention can also be expressed from a nucleic acid construct administered to the individual employing any suitable mode of administration, described hereinabove (i.e., in-vivo gene therapy). In one embodiment, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex-vivo gene therapy).

In one embodiment, plant expression vectors are used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

Various methods, in some embodiments, can be used to introduce the expression vector of the present invention into the host cell system. In some embodiments, such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In some embodiments, transformed cells are cultured under effective conditions, which allow for the expression of high amounts of recombinant polypeptide. In some embodiments, effective culture conditions include, but are not limited to, effective media, bioreactor, temperature, pH and oxygen conditions that permit protein production. In one embodiment, an effective medium refers to any medium in which a cell is cultured to produce the recombinant polypeptide of the present invention. In some embodiments, a medium typically includes an aqueous solution having assimilable carbon, nitrogen and phosphate sources, and appropriate salts, minerals, metals and other nutrients, such as vitamins. In some embodiments, cells of the present invention can be cultured in conventional fermentation bioreactors, shake flasks, test tubes, microtiter dishes and petri plates. In some embodiments, culturing is carried out at a temperature, pH and oxygen content appropriate for a recombinant cell. In some embodiments, culturing conditions are within the expertise of one of ordinary skill in the art.

In some embodiments, depending on the vector and host system used for production, resultant polypeptides of the present invention either remain within the recombinant cell, secreted into the fermentation medium, secreted into a space between two cellular membranes, such as the periplasmic space in E. coli; or retained on the outer surface of a cell or viral membrane.

In one embodiment, following a predetermined time in culture, recovery of the recombinant polypeptide is effected.

In one embodiment, the phrase “recovering the recombinant polypeptide” used herein refers to collecting the whole fermentation medium containing the polypeptide and need not imply additional steps of separation or purification.

In one embodiment, polypeptides of the present invention are purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

In one embodiment, to facilitate recovery, the expressed coding sequence can be engineered to encode the polypeptide of the present invention and fused cleavable moiety. In one embodiment, a fusion protein can be designed so that the polypeptide can be readily isolated by affinity chromatography; e.g., by immobilization on a column specific for the cleavable moiety. In one embodiment, a cleavage site is engineered between the polypeptide and the cleavable moiety and the polypeptide can be released from the chromatographic column by treatment with an appropriate enzyme or agent that specifically cleaves the fusion protein at this site [e.g., see Booth et al., Immunol. Lett. 19:65-70 (1988); and Gardella et al., J. Biol. Chem. 265:15854-15859 (1990)].

In one embodiment, the polypeptide of the present invention is retrieved in “substantially pure” form.

In one embodiment, the phrase “substantially pure” refers to a purity that allows for the effective use of the protein in the applications described herein.

In one embodiment, the polypeptide of the present invention can also be synthesized using in vitro expression systems. In one embodiment, in vitro synthesis methods are well known in the art and the components of the system are commercially available.

In one embodiment, a “pharmaceutical composition” refers to a preparation of one or more of the active ingredients or long-acting ADH/KRED and/or KREDs described herein with other chemical components such as physiologically suitable carriers and excipients. The purpose of a pharmaceutical composition is to facilitate administration of a compound to an organism.

In one embodiment, “active ingredient” refers to the polypeptide sequence of interest, KREDs and/or long-acting ADH/KRED, which is accountable for the biological effect as described herein.

In one embodiment, the present invention provides combined preparations. In one embodiment, “a combined preparation” defines especially a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. In one embodiment, the combined preparation can be varied, e.g., in order to cope with the needs of a patient subpopulation to be treated or the needs of the single patient which different needs can be due to a particular disease, severity of a disease, age, sex, or body weight as can be readily made by a person skilled in the art.

In one embodiment, the phrases “physiologically acceptable carrier” and “pharmaceutically acceptable carrier” which be interchangeably used refer to a carrier or a diluent that does not cause significant irritation to an organism and does not abrogate the biological activity and properties of the administered compound. An adjuvant is included under these phrases. In one embodiment, one of the ingredients included in the pharmaceutically acceptable carrier can be for example polyethylene glycol (PEG), a biocompatible polymer with a wide range of solubility in both organic and aqueous media (Mutter et al. (1979).

In one embodiment, “excipient” refers to an inert substance added to a pharmaceutical composition to further facilitate administration of an active ingredient. In one embodiment, excipients include calcium carbonate, calcium phosphate, various sugars and types of starch, cellulose derivatives, gelatin, vegetable oils and polyethylene glycols.

Techniques for formulation and administration of drugs are found in “Remington's Pharmaceutical Sciences,” Mack Publishing Co., Easton, Pa., latest edition, which is incorporated herein by reference.

In one embodiment, suitable routes of administration, for example, include oral, rectal, transmucosal, transnasal, intestinal or parenteral delivery, including intramuscular, subcutaneous and intramedullary injections as well as intrathecal, direct intraventricular, intravenous, intraperitoneal, intranasal, or intraocular injections.

In one embodiment, the preparation is administered in a local rather than systemic manner, for example, via injection of the preparation directly into a specific region of a patient's body.

Various embodiments of dosage ranges are contemplated by this invention. The dosage of the polypeptide of the present invention, in one embodiment, is in the range of 0.05-800 mg/day. The dosage of the polypeptide of the present invention, in one embodiment, is in the range of 0.05-10 g/day. The dosage of the polypeptide of the present invention, in one embodiment, is in the range of 5-150 g/day. The dosage of the polypeptide of the present invention, in one embodiment, is in the range of 1-30 g/day. The dosage of the polypeptide of the present invention, in one embodiment, is in the range of 0.05-80 mg/day. In another embodiment, the dosage is in the range of 0.05-50 mg/day. In another embodiment, the dosage is in the range of 0.1-20 mg/day. In another embodiment, the dosage is in the range of 0.1-10 mg/day. In another embodiment, the dosage is in the range of 0.1-5 mg/day. In another embodiment, the dosage is in the range of 0.5-5 mg/day. In another embodiment, the dosage is in the range of 0.5-50 mg/day. In another embodiment, the dosage is in the range of 5-80 mg/day. In another embodiment, the dosage is in the range of 35-65 mg/day. In another embodiment, the dosage is in the range of 35-65 mg/day. In another embodiment, the dosage is in the range of 20-60 mg/day. In another embodiment, the dosage is in the range of 40-60 mg/day. In another embodiment, the dosage is in a range of 45-60 mg/day. In another embodiment, the dosage is in the range of 40-60 mg/day. In another embodiment, the dosage is in a range of 60-120 mg/day. In another embodiment, the dosage is in the range of 120-240 mg/day. In another embodiment, the dosage is in the range of 40-60 mg/day. In another embodiment, the dosage is in a range of 240-400 mg/day. In another embodiment, the dosage is in a range of 45-60 mg/day. In another embodiment, the dosage is in the range of 15-25 mg/day. In another embodiment, the dosage is in the range of 5-10 mg/day. In another embodiment, the dosage is in the range of 55-65 mg/day.

In one embodiment, the dosage is 20 mg/day. In another embodiment, the dosage is 30 mg/day. In another embodiment, the dosage is 40 mg/day. In another embodiment, the dosage is 50 mg/day. In another embodiment, the dosage is 60 mg/day. In another embodiment, the dosage is 70 mg/day. In another embodiment, the dosage is 80 mg/day. In another embodiment, the dosage is 90 mg/day. In another embodiment, the dosage is 100 mg/day.

Oral administration, in one embodiment, comprises a unit dosage form comprising tablets, capsules, lozenges, chewable tablets, suspensions, emulsions and the like. Such unit dosage forms comprise a safe and effective amount of the desired compound, or compounds, each of which is in one embodiment, from about 1 or 10 mg to about 50 mg/70 kg, or in another embodiment, about 1 or 10 mg to about 10 g/70 kg. Such unit dosage forms comprise a safe and effective amount of the desired compound, or compounds, each of which is in one embodiment, from about 0.7 or 3.5 mg to about 280 mg/70 kg, or in another embodiment, about 0.5 or 10 mg to about 210 mg/70 kg. The pharmaceutically-acceptable carriers suitable for the preparation of unit dosage forms for peroral administration are well-known in the art. In some embodiments, tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. In one embodiment, glidants such as silicon dioxide can be used to improve flow characteristics of the powder-mixture. In one embodiment, coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. In some embodiments, the selection of carrier components depends on secondary considerations like taste, cost, and shelf stability, which are not critical for the purposes of this invention, and can be readily made by a person skilled in the art.

In one embodiment, the oral dosage form comprises predefined release profile. In one embodiment, the oral dosage form of the present invention comprises an extended release tablets, capsules, lozenges or chewable tablets. In one embodiment, the oral dosage form of the present invention comprises a slow release tablets, capsules, lozenges or chewable tablets. In one embodiment, the oral dosage form of the present invention comprises an immediate release tablets, capsules, lozenges or chewable tablets. In one embodiment, the oral dosage form is formulated according to the desired release profile of the pharmaceutical active ingredient as known to one skilled in the art.

Peroral compositions, in some embodiments, comprise liquid solutions, emulsions, suspensions, and the like. In some embodiments, pharmaceutically-acceptable carriers suitable for preparation of such compositions are well known in the art. In some embodiments, liquid oral compositions comprise from about 0.012% to about 0.933% of the desired compound or compounds, or in another embodiment, from about 0.033% to about 0.7%.

In some embodiments, compositions for use in the methods of this invention comprise solutions or emulsions, which in some embodiments are aqueous solutions or emulsions comprising a safe and effective amount of the compounds of the present invention and optionally, other compounds, intended for topical intranasal administration. In some embodiments, h compositions comprise from about 0.01% to about 10.0% w/v of a subject compound, more preferably from about 0.1% to about 2.0, which is used for systemic delivery of the compounds by the intranasal route.

In another embodiment, the pharmaceutical compositions are administered by intravenous, intra-arterial, or intramuscular injection of a liquid preparation. In some embodiments, liquid formulations include solutions, suspensions, dispersions, emulsions, oils and the like. In one embodiment, the pharmaceutical compositions are administered intravenously, and are thus formulated in a form suitable for intravenous administration. In another embodiment, the pharmaceutical compositions are administered intra-arterially, and are thus formulated in a form suitable for intra-arterial administration. In another embodiment, the pharmaceutical compositions are administered intramuscularly, and are thus formulated in a form suitable for intramuscular administration.

Further, in another embodiment, the pharmaceutical compositions are administered topically to body surfaces, and are thus formulated in a form suitable for topical administration. Suitable topical formulations include gels, ointments, creams, lotions, drops and the like. For topical administration, the compounds of the present invention are combined with an additional appropriate therapeutic agent or agents, prepared and applied as solutions, suspensions, or emulsions in a physiologically acceptable diluent with or without a pharmaceutical carrier.

In one embodiment, pharmaceutical compositions of the present invention are manufactured by processes well known in the art, e.g., by means of conventional mixing, dissolving, granulating, dragee-making, levigating, emulsifying, encapsulating, entrapping or lyophilizing processes.

In one embodiment, pharmaceutical compositions for use in accordance with the present invention is formulated in conventional manner using one or more physiologically acceptable carriers comprising excipients and auxiliaries, which facilitate processing of the active ingredients into preparations which, can be used pharmaceutically. In one embodiment, formulation is dependent upon the route of administration chosen.

In one embodiment, injectables, of the invention are formulated in aqueous solutions. In one embodiment, injectables, of the invention are formulated in physiologically compatible buffers such as Hank's solution, Ringer's solution, or physiological salt buffer. In some embodiments, for transmucosal administration, penetrants appropriate to the barrier to be permeated are used in the formulation. Such penetrants are generally known in the art.

In one embodiment, the preparations described herein are formulated for parenteral administration, e.g., by bolus injection or continuous infusion. In some embodiments, formulations for injection are presented in unit dosage form, e.g., in ampoules or in multidose containers with optionally, an added preservative. In some embodiments, compositions are suspensions, solutions or emulsions in oily or aqueous vehicles, and contain formulatory agents such as suspending, stabilizing and/or dispersing agents.

The compositions also comprise, in some embodiments, preservatives, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcystine, sodium metabisulfote and others; aromatic agents; viscosity adjustors, such as polymers, including cellulose and derivatives thereof; and polyvinyl alcohol and acid and bases to adjust the pH of these aqueous compositions as needed. The compositions also comprise, in some embodiments, local anesthetics or other actives. The compositions can be used as sprays, mists, drops, and the like.

In some embodiments, pharmaceutical compositions for parenteral administration include aqueous solutions of the active preparation in water-soluble form. Additionally, suspensions of the active ingredients, in some embodiments, are prepared as appropriate oily or water based injection suspensions. Suitable lipophilic solvents or vehicles include, in some embodiments, fatty oils such as sesame oil, or synthetic fatty acid esters such as ethyl oleate, triglycerides or liposomes. Aqueous injection suspensions contain, in some embodiments, substances, which increase the viscosity of the suspension, such as sodium carboxymethyl cellulose, sorbitol or dextran. In another embodiment, the suspension also contain suitable stabilizers or agents which increase the solubility of the active ingredients to allow for the preparation of highly concentrated solutions.

In another embodiment, the active compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid).

In another embodiment, the pharmaceutical composition delivered in a controlled release system is formulated for intravenous infusion, implantable osmotic pump, transdermal patch, liposomes, or other modes of administration. In one embodiment, a pump is used (see Langer, supra; Sefton, CRC Crit. Ref. Biomed. Eng. 14:201 (1987); Buchwald et al., Surgery 88:507 (1980); Saudek et al., N. Engl. J. Med 321:574 (1989). In another embodiment, polymeric materials can be used. In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

In some embodiments, the active ingredient is in powder form for constitution with a suitable vehicle, e.g., sterile, pyrogen-free water based solution, before use. Compositions are formulated, in some embodiments, for atomization and inhalation administration. In another embodiment, compositions are contained in a container with attached atomizing means.

In one embodiment, the preparation of the present invention is formulated in rectal compositions such as suppositories or retention enemas, using, e.g., conventional suppository bases such as cocoa butter or other glycerides.

In some embodiments, pharmaceutical compositions suitable for use in context of the present invention include compositions wherein the active ingredients are contained in an amount effective to achieve the intended purpose. In some embodiments, a therapeutically effective amount means an amount of active ingredients effective to prevent, alleviate or ameliorate symptoms of disease or prolong the survival of the subject being treated.

In one embodiment, determination of a therapeutically effective amount is well within the capability of those skilled in the art.

The compositions also comprise preservatives, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcystine, sodium metabisulfote and others; aromatic agents; viscosity adjustors, such as polymers, including cellulose and derivatives thereof; and polyvinyl alcohol and acid and bases to adjust the pH of these aqueous compositions as needed. The compositions also comprise local anesthetics or other actives. The compositions can be used as sprays, mists, drops, and the like.

Some examples of substances which can serve as pharmaceutically-acceptable carriers or components thereof are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the Tween™ brand emulsifiers; wetting agents, such sodium lauryl sulfate; coloring agents; flavoring agents; tableting agents, stabilizers; antioxidants; preservatives; pyrogen-free water; isotonic saline; and phosphate buffer solutions. The choice of a pharmaceutically-acceptable carrier to be used in conjunction with the compound is basically determined by the way the compound is to be administered. If the subject compound is to be injected, in one embodiment, the pharmaceutically-acceptable carrier is sterile, physiological saline, with a blood-compatible suspending agent, the pH of which has been adjusted to about 7.4.

In addition, the compositions further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCI., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents (e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants.

Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, cellulose (e.g. Avicel™, RC-591), tragacanth and sodium alginate; typical wetting agents include lecithin and polyethylene oxide sorbitan (e.g. polysorbate 80). Typical preservatives include methyl paraben and sodium benzoate. In another embodiment, peroral liquid compositions also contain one or more components such as sweeteners, flavoring agents and colorants disclosed above.

The compositions also include incorporation of the active material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance.

Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

In some embodiments, compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. In another embodiment, the modified compounds exhibit substantially longer half-lives in blood following intravenous injection than do the corresponding unmodified compounds. In one embodiment, modifications also increase the compound's solubility in aqueous solution, eliminate aggregation, enhance the physical and chemical stability of the compound, and greatly reduce the immunogenicity and reactivity of the compound. In another embodiment, the desired in vivo biological activity is achieved by the administration of such polymer-compound abducts less frequently or in lower doses than with the unmodified compound.

In some embodiments, preparation of effective amount or dose can be estimated initially from in vitro assays. In one embodiment, a dose can be formulated in animal models and such information can be used to more accurately determine useful doses in humans.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975) “The Pharmacological Basis of Therapeutics”, Ch. 1 p. 1].

In one embodiment, depending on the severity and responsiveness of the condition to be treated, dosing can be of a single or a plurality of administrations, with course of treatment lasting from several days to several weeks or until cure is effected or diminution of the disease state is achieved.

In one embodiment, the amount of a composition to be administered will, of course, be dependent on the subject being treated, the severity of the affliction, the manner of administration, the judgment of the prescribing physician, etc.

In one embodiment, compositions including the preparation of the present invention formulated in a compatible pharmaceutical carrier are also be prepared, placed in an appropriate container, and labeled for treatment of an indicated condition.

In one embodiment, compositions of the present invention are presented in a pack or dispenser device, such as an FDA approved kit, which contain one or more unit dosage forms containing the active ingredient. In one embodiment, the pack, for example, comprise metal or plastic foil, such as a blister pack. In one embodiment, the pack or dispenser device is accompanied by instructions for administration. In one embodiment, the pack or dispenser is accommodated by a notice associated with the container in a form prescribed by a governmental agency regulating the manufacture, use or sale of pharmaceuticals, which notice is reflective of approval by the agency of the form of the compositions or human or veterinary administration. Such notice, in one embodiment, is labeling approved by the U.S. Food and Drug Administration for prescription drugs or of an approved product insert.

In one embodiment, it will be appreciated that the polypeptides of the present invention can be provided to the individual with additional active agents to achieve an improved therapeutic effect as compared to treatment with each agent by itself. In another embodiment, measures (e.g., dosing and selection of the complementary agent) are taken to adverse side effects which are associated with combination therapies.

As used herein, the singular forms “a”, “an”, and “the” include plural forms unless the context clearly dictates otherwise. Thus, for example, reference to “a therapeutic agent” includes reference to more than one therapeutic agent.

Unless specifically stated or obvious from context, as used herein, the term “or” is understood to be inclusive.

The term “including” is used herein to mean, and is used interchangeably with, the phrase “including but not limited to.”

As used herein, the terms “comprises,” “comprising,” “containing,” “having” and the like can have the meaning ascribed to them in U.S. patent law and can mean “includes,” “including,” and the like; “consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In another embodiment, the term “comprise” includes the term “consist”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Study Design—Behavioral Studies-Activity Box

On Day −3, mice were placed in the activity box for 10 minutes (min) for habituation. 2 hours (h) later all mice received an IP injection of 0.25 ml saline and began 10 min habituation.

On Day −2 mice received 0.25 ml saline IP, returned to their home cage, and 10 min later their locomotor activity was evaluated for 30 min in the Activity Box. This individual baseline measurement was used for comparison to the behaviour following treatment.

On Day 1 mice treated with the Vehicle, ADH or water for injection (WFI) IV 20 min before testing in the Activity Box. 10 min before testing, mice received 0.5 g/kg of EtOH or Saline IP.

### General Activity Box (Open Field) Procedure:

30 min test; 30 min baseline. The percent difference between test and baseline was calculated per individual mouse. Distance travelled was measured as the primary variable.

### Example 1: Effect of EtOH Dose (0.05 g/Ml-0.2 g/Ml) on Open Field Activity (Activity Box)

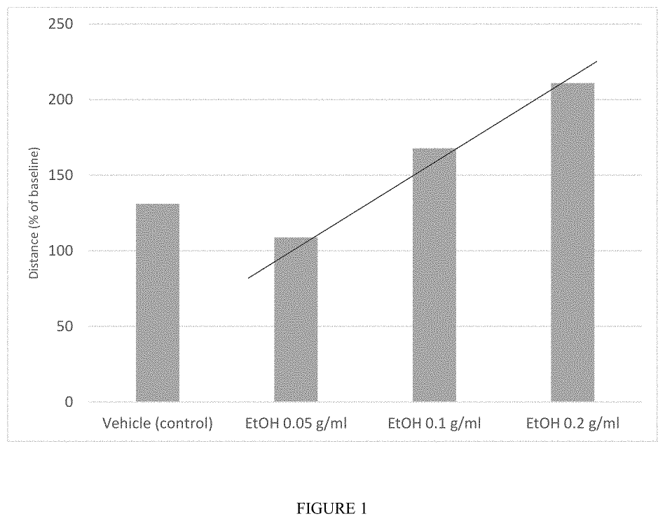

As shown in FIG. 1, there is a direct correlation between the amount of alcohol-EtOH administered and the reduction in performance of the treated mice. Distance traveled was measured along 30 min and compared to individual mouse baseline, 10 min following EtOH IP administration (10 ml/kg solution).

### Example 2: The Effect of EtOH is Quenched by Alcohol Dehydrogenase (KRED) on Open Field Activity (Activity Box)

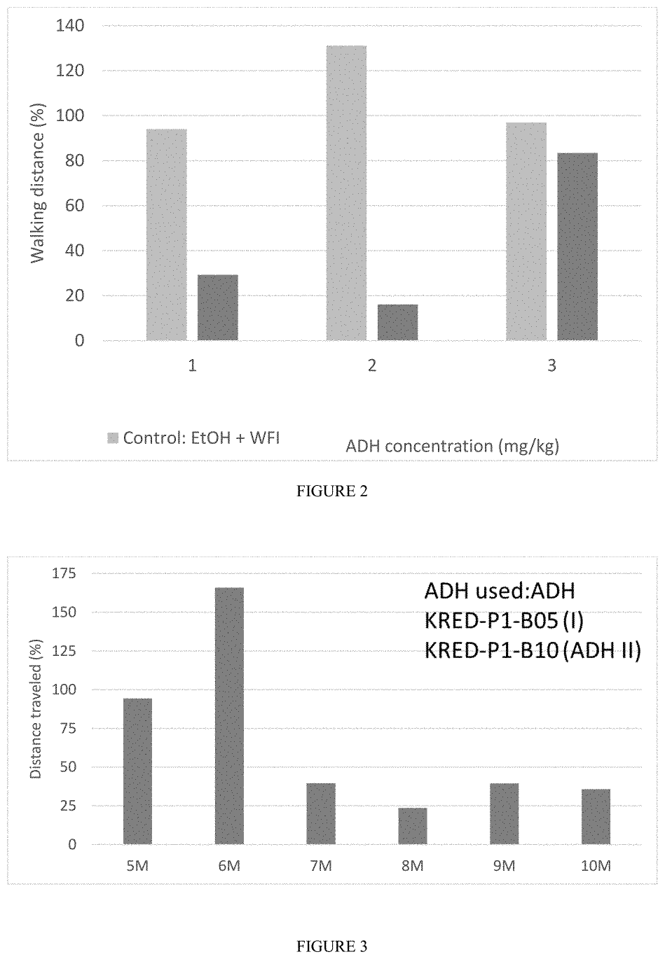

FIG. 2 further demonstrates the distance traveled in activity Box test for 30 min. Specifically, Effect of EtOH (0.5 g/kg) and ADH (10, 100 and 500 mg/kg) (cycles 1&2). Measurements of distance traveled in Activity Box tests during a 30 min of trial. ADH used: 500 mg/kg: KRED-P1-A04; 100/10 mg/kg: KRED-P1-A12. As FIG. 2 clearly demonstrates the effect of EtOH on activity was inhibited and/or quenched by KRED.

### Example 3: The Duration of the Quenching Effect of Alcohol Dehydrogenase (KRED) on EtOH Administration and the Preventive Effect of Alcohol Dehydrogenase (KRED) on Later EtOH Administration as Measured in an Open Field Activity (Activity Box)

Table 1, hereinbelow summarizes the experimental protocol showing that a KRED as described herein both inhibits and/or quenches EtOH effects on the CNS and that KRED can prevent the devastating effects of EtOH when administered prior to consumption of alcohol.

TABLE 1

| Group | 20 min before testing | 10 min before testing |
|---|---|---|
| 5M | Water for Injection (WFI) | Saline-IP |
| 6M | Water for Injection (WFI) | EtOH 0.1 g/kg-IP |
| 7M | ADH II, 50 mg/kg (IV) | EtOH 0.1 g/kg-IP |
| | ADH I, 50 mg/kg (IV) | |
| 8M | ADH II, 100 mg/kg (IV) | EtOH 0.1 g/kg-IP |
| | ADH I, 100 mg/kg (IV) | |
| 9M | EtOH 0.1 g/kg-IP | ADH II, 50 mg/kg (IV) |
| | | ADH I, 50 mg/kg (IV) |
| 10M | EtOH 0.1 g/kg-IP | ADH II, 100 mg/kg (IV) |
| | | ADH I, 100 mg/kg (IV) |

FIG. 3 further demonstrates that the KREDs of the invention not only rescue EtOH devastating CNS effects but rather have a preventive-protecting role against EtOH devastating CNS effects. Specifically, the ADH used were KRED-P1-B05 ADH (I) and KRED-P1-B10 (ADH II). Activity Box Test shows total distance traveled (15 min). It is important to notice that in FIG. 3 the value correlates with soberness (less effect on the CNS).

Thus according to the present results a dose of 0.5 g/kg EtOH led to mild but not dramatic increase in the distance (velocity) of mice moving in the Activity Box. Dose of 1 g/kg EtOH led to significant increase in movement. The vehicle (media), per-se, used for administering ADH had no effect at all. Interestingly, ADH decreased the movement of the mice considerably in a dose-dependent manner, with 100 mg/kg dose presenting a plateau value.

Both, ADH administration before/after EtOH abuse led to similar decrease in the measured distance.

### Example 4: EtOH Rescue and the Beam Test

The beam walking test was designed to assess motor coordination deficits following EtOH administration. The beam used was: 1 cm flat wood, 80 cm in length, 1 meter above the floor.

Acclimation of at least 30 min. Mice were habituated to the goal box for 2 min, and then placed at a distance of 10 cm from the goal box and allowed to traverse the beam to the goal box.

Upon successful traversal of the beam to the goal box, mice were placed at increasing distances of 30, 50, and 80 cm and trained to traverse the beam for two consecutive days. Mice able to traverse the full 80 cm length to the goal box within 60 sec were considered to fit for the study.

Baseline measurement: After 2 training days, mice were tested to obtain individual “baseline value”. Each animal was given 3 consecutive trials until it manages to cross the whole beam. The elapsed time to reach the home cage during the 3 sessions was recorded.

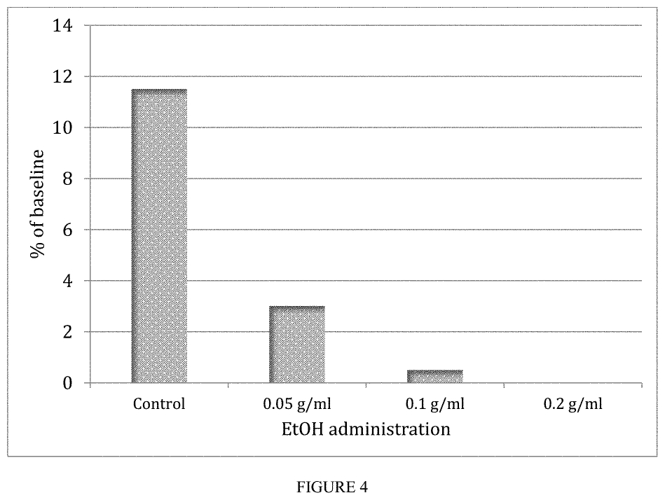

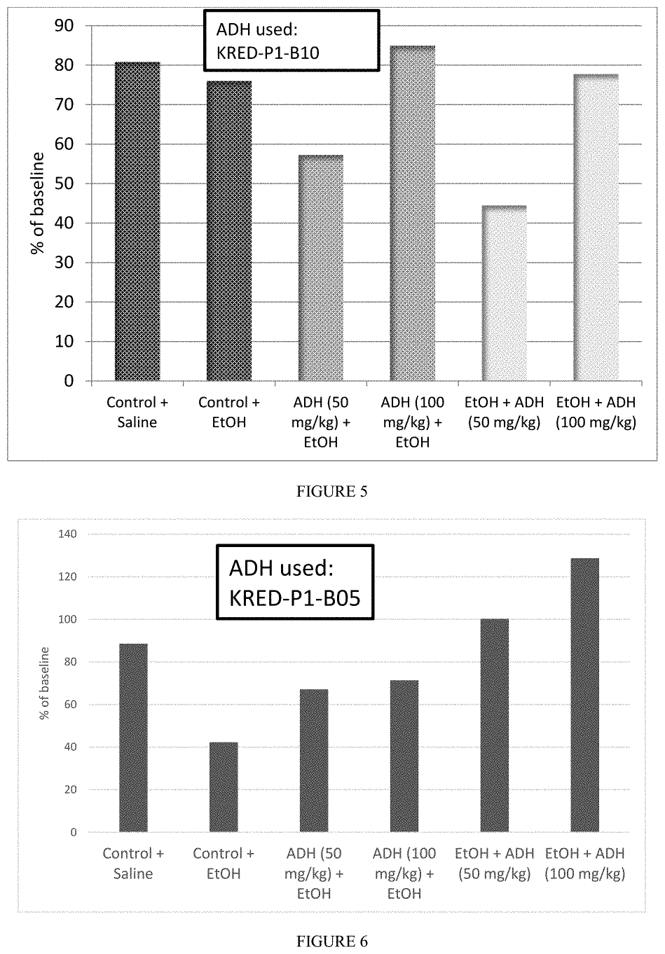

FIG. 4 demonstrates the inverse correlation between distances travelled on a beam and EtOH consumption by the mice. FIG. 5 demonstrates that KREDs such as KRED-P1-B10 of the invention rescue mice from immediately falling from the beam and/or alternatively enabling the mice to travel longer distances after EtOH consumption (such as 50 mg/kg ADH). ADH used:

Moreover, the experiments conducted also showed that ADH I (KRED-P1-B05) had a higher protective activity and was found to be effective at inhibiting EtOH effects both before and after administration EtOH (FIG. 6, the value of bars correlates with soberness).

### Example 5: In-Vivo Human Study: Evaluation of the Effect of ADH on EtOH Concentration in Human Plasma

In this study 3 volunteers were tested for the effects of KRED-ADH 30 minutes after consumption of EtOH. Total amount consumed of the KRED: In the first experiment, to each 280μ1 of plasma sample, 10μ1 of enzyme stock were added and after about 1 minute, 10 μl of EtOH stock were added to the plasma/enzyme solution. In the second experiment, to each 130μ1 of plasma sample, 5 μl of enzyme stock were added and after 1 minute, 5 μl of EtOH stock were added to the plasma/enzyme solution.

Analyses were performed using Analox AM1 EtOH analyzer at the 30 min time point (after EtOH consumption).

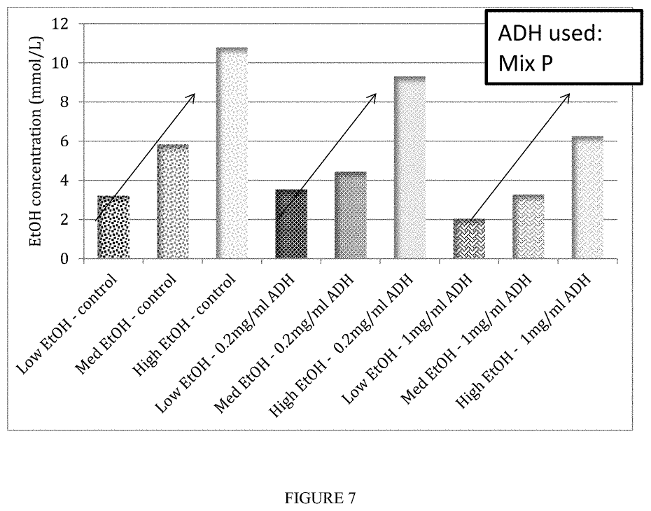

Significant decrease in EtOH concentration was observed in the high EtOH solution (2 mg/ml), using the medium and high ADH concentrations, compared to the control treated group (FIG. 7). A significant reduction was observed in the medium EtOH solution (1 mg/ml), but only when using high ADH concentration (FIG. 7). The optimal EtOH dose was evaluated and tested in combination with two ADH concentrations against a control reference.

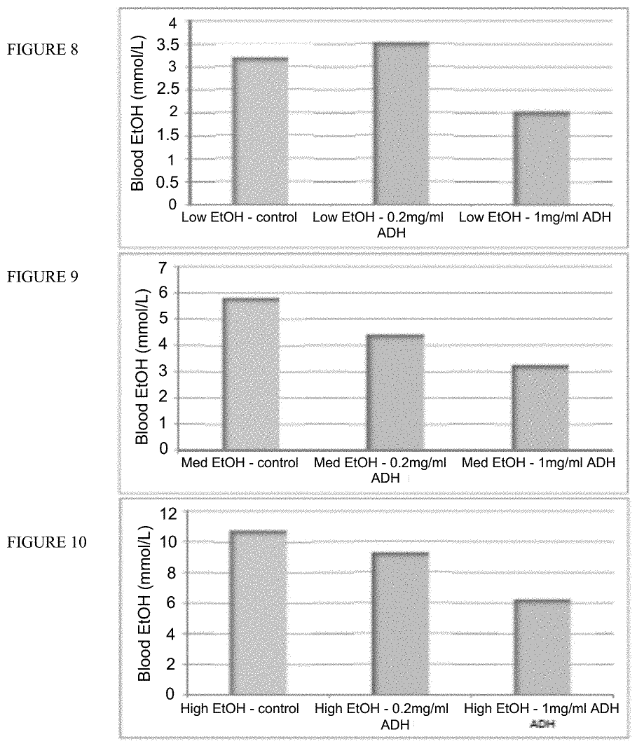

Moreover, evaluation of the effect of ADH on EtOH concentration in human plasma (3 volunteers 30 min after ADH addition) revealed that KREDs of the invention lowered blood EtOH level after administering low EtOH administration (0.5 mg/mL). Specifically, a reduction of 37% using 1 mg/ml ADH was recorded (FIG. 8).

KREDs of the invention also lowered blood EtOH level after administering medium EtOH administration (1 mg/mL). Specifically, a reduction of 44% using 1 mg/ml ADH was recorded (FIG. 9).

KREDs of the invention also lowered blood EtOH level after administering high EtOH administration (2 mg/mL). Specifically, a reduction of 42% using 2 mg/ml ADH was recorded (FIG. 10).

### Example 7: Evaluation of the Effect of ADHs on Acute EtOH Intoxication at Various Doses in Mice

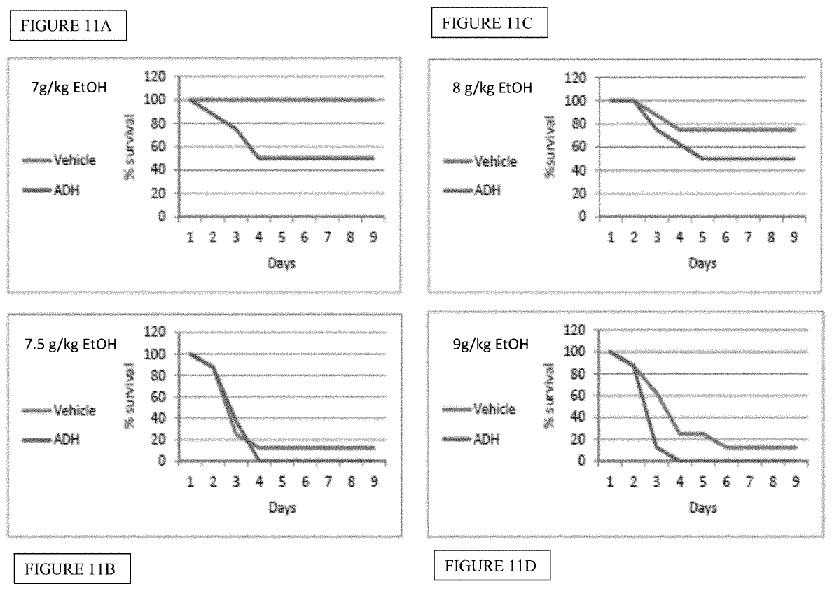

In the present study the lethal effect of high doses of EtOH was quenches rescued. Specifically, as shown in FIG. 11 A-D KREDs of the invention rescued the steep physiological deterioration following acute administration of EtOH. Body weight was not affected by ADH treatment (Mix P (of ADHes at 50 mg/kg).

All EtOH treated animals displayed signs of EtOH intoxication such as DMA (Decreased Motor Activity), dyspnea (shortness of breath) and ataxia (lack of voluntary coordination of muscle movements).

In EtOH blood analysis, at the two lower EtOH doses of 7 and 7.5 g/kg—ADH treated groups exhibited a trend towards lowering EtOH concentrations. However, at the two higher EtOH doses of 8 and 9 g/kg—ADH treated groups showed higher EtOH concentrations. An interaction between the human ADH and an endogenic mouse factor in the blood may explain this phenomenon.

The conventional view is that alcohol metabolism is carried out by ADH1 in the liver. However, it has been suggested that another pathway plays an important role in alcohol metabolism, especially when the level of blood ethanol is high or when drinking is chronic.

In mice, generally saying change in liver ADH activity depended on both dose and time.

However, change in class I ADH (ADH1) content depended on dose alone and the change of class III ADH (ADH3) content depended on both dose and time.

1. A protein comprising an amino acid sequence encoding ADH/KRED bound to at least one long-acting molecule.

2. The protein of claim 1, wherein said at least one long-acting molecule is bound to the amino terminus of said amino acid sequence encoding ADH/KRED, to the carboxy terminus of said amino acid sequence encoding ADH/KRED, or both.

3. (canceled)

4. (canceled)

5. (canceled)

6. (canceled)

7. (canceled)

8. A pharmaceutical composition, comprising the protein of claim 1 and a pharmaceutically acceptable carrier.

9. The pharmaceutical composition of claim 8, comprising 10 mg to 100 g of said protein.

10. (canceled)

11. (canceled)

12. (canceled)

13. A method for lowering blood alcohol in a subject in need thereof, comprising administering to said subject a composition comprising an effective amount of the protein of claim 1, thereby lowering blood alcohol in a subject in need thereof.

14. (canceled)

15. The method of claim 13, wherein said effective amount is 1 to 500 mg/kg (body weight).

16. The method of claim 13, wherein said subject in need thereof has a blood EtOH concentration of above 0.001% by blood volume.

17. (canceled)

18. (canceled)

19. (canceled)

20. (canceled)

21. (canceled)

22. (canceled)

23. (canceled)

24. (canceled)

25. (canceled)

26. A method for preventing a symptom or a risk arising from alcohol consumption in a subject in need thereof, comprising administering to said subject a composition comprising an effective amount of KRED-P1-A04, KRED-P1-A12, KRED-P1-B05, KRED-P1-B10, or any combination thereof, thereby preventing a symptom or a risk arising from alcohol consumption in a subject in need thereof.

27. The method of claim 26, wherein said subject in need thereof is a subject destined to consume alcohol.

28. The method of claim 26, wherein said subject in need thereof is a subject destined to consume alcohol within 1 to 90 minutes.

29. (canceled)

30. (canceled)

31. The method of claim 26, wherein said subject is afflicted with alcoholism.

32. The method of claim 31, wherein said administering is administering before alcohol consumption, after alcohol consumption, or both before and after alcohol consumption.

33. The method of claim 31, wherein before, after, or both comprise 1 to 90 minutes.

34. (canceled)

35. (canceled)

36. (canceled)

37. (canceled)

38. (canceled)

39. (canceled)

40. (canceled)

41. (canceled)

42. A pharmaceutical composition, comprising 10 mg to 100 g of KRED-P1-A04, KRED-P1-A12, KRED-P1-B05, KRED-P1-B10, or any combination thereof and a pharmaceutically acceptable carrier.

43. (canceled)

44. The pharmaceutical composition of claim 42, comprising 50 mg to 1 g of KRED-P1-A04, KRED-P1-A12, KRED-P1-B05, KRED-P1-B10, or any combination thereof.

45. (canceled)

46. (canceled)

47. (canceled)

48. The pharmaceutical composition of claim 42, for use in a subject having a blood ethanol concentration of above 0.001% by blood volume.

49. (canceled)

50. (canceled)

51. The pharmaceutical composition of claim 42, for preventing a symptom or a risk arising from alcohol consumption in a subject in need thereof.

52. (canceled)

53. (canceled)

54. (canceled)

55. (canceled)

56. (canceled)

57. (canceled)

58. (canceled)

59. (canceled)

60. (canceled)


SEQUENCE LISTING (1 sequence)

SEQ ID NO: 1 - protein, 283 aa, Schizosaccharomyces pombe
  PEPTIDE (1)..(283)
MAKNFSNVEYPAPPPAHTKNESLQVLDLFKLNGKVASITGSSSGIGYALAEAFAQVGADVAIWYNSHDATGKAEALAKKYGVKVKAYKANVSSSDAVKQTIEQQIKDFGHLDIVVANAGIPWTKGAYIDQDDDKHFDQVVDVDLKGVGYVAKHAGRHFRERFEKEGKKGALVFTASMSGHIVNVPQFQATYNAAKAGVRHFAKSLAVEFAPFARVNSVSPGYINTEISDFVPQETQNKWWSLVPLGRGGETAELVGAYLFLASDAGSYATGTDIIVDGGYTLP